(19) United States
(12) Patent Application Publication
(10) Pub. No.: US 2025/0369907 A1
(43) Pub. Date: Dec. 4, 2025

(54) BATTERY CONTAINMENT DEVICES FOR THERMAL MEASUREMENTS

(71) Applicant: TA Instruments-Waters LLC, New Castle, DE (US)

(72) Inventors: Nathan Roehr, Wilmington, DE (US); Robert L. Danley, Collingswood, NJ (US); Mark E. Cunningham, Rising Sun, MD (US); Xiaoping Hu, New Castle, DE (US); Matthew R. Lewis, Middletown, DE (US); Peter A. Caulfield, Downingtown, DE (US); Kirk Miller, Middletown, DE (US)

(21) Appl. No.: 19/224,157

(22) Filed: May 30, 2025

Related U.S. Application Data
(60) Provisional application No. 63/653,572, filed on May 30, 2024.
(60) Provisional application No. 63/725,165, filed on Nov. 26, 2024.
(60) Provisional application No. 63/754,390, filed on Feb. 5, 2025.

(51) Int. Cl. G01N 25/48

(52) U.S. Cl. CPC G01N 25/4813

(57) ABSTRACT

A battery material containment device comprises a lid; a housing case that is constructed and arranged to hold a battery material during a thermal analysis operation, the housing case including a threaded region for receiving and removably coupling with the lid to form a gas-tight seal and securing the battery material in the housing case; at least one vent for providing a gas flow path for the release of gasses generated by the battery material in the housing case during the thermal analysis operation; and a heat flow pathway between the battery material and a sensor that is maintained during the thermal analysis operation.

### RELATED APPLICATIONS

This application claims priority to U.S. Provisional Patent No. 63/754,390 filed Feb. 5, 2025 entitled “Battery Containment Devices for Thermal Measurements,” the contents of which are incorporated by reference herein in their entirety. This application also claims priority to U.S. Provisional Patent No. 63/653,572 filed May 30, 2024 entitled “Device for Use in Testing and Analysis of Battery Materials,” and U.S. Provisional Patent No. 63/725,165 filed Nov. 26, 2024 entitled “Battery Material Containment Device Allowing Various Analyses Under Extreme Conditions,” the contents of each of which are incorporated by reference herein in their entirety.

### FIELD OF THE INVENTION

The disclosed technology generally relates to battery testing and analysis. More particularly, the disclosed technology relates to devices, systems, and methods for performing coin cell temperature calorimetry measurements.

### BACKGROUND

Battery manufacturers are constantly desiring higher energy density chemistries from lithium-ion and other battery technology as well as improved safety from battery cells. Safety testing of a complete cell is generally performed after reaching pilot-scale manufacturing quantities. Conventional workflows must scale cell chemistries to larger formats before testing full cells. However, it is time consuming and expensive to change the chemistry after cell formulation has scaled to the pilot level.

Coin cells generally include a small, polypropylene or other plastic ring or gasket designed to ensure that the battery compartment is sealed properly. Safety testing of coin cells includes calorimetry measurements taken across a wide temperature range, from sub-ambient to 600° C. or more. The thermal analysis of battery chemistries assist scientists and engineers understand the safety and durability of new battery materials. However, during thermal testing at temperatures from sub-ambient to 150° C., some coin cells such as 2032 coin cells housing a Li-ion battery can experience deformation and yielding of the coin cell lid and cup as internal pressure builds, which can change the contact resistance between the coin cell and calorimetry sensor, e.g., DSC sensor illustrated herein. Laterally, the coin cell's lid and cup will begin to separate from each other as the sealing gasket, typically an O-ring formed of polypropylene, melts. Continuing to heat above 150° C. will likely result in the coin cell rapidly disassembling and the battery materials and CC internals jettisoning away from any sensor platform. The deformation and yielding of the coin cell's case can preclude or obfuscate meaningful thermal analysis as the thermal contact between sample and sensor dynamically changes and the yielding and deformation themselves cause undesirable heat flows. Furthermore, rapid disassembly can prevent thermal analysis above the sealing gasket's melting point. For adequate calorimetric measurement across the entire temperature range, the battery material capsule should stay in contact with the calorimeter sensor during gas venting, and not undergo yielding, or disassembly across the temperature range of interest. In other cases where gas venting does not occur, i.e., the gas is contained in the cell, it is also desirable not to undergo yielding or disassembly.

There is a desire by battery cell manufacturers evaluating the safety of chemistries at the coin cell level during formulation before scaling to pilot-level or mass production manufacturing quantities. In addition, there is a desire to perform adequate calorimetric measurements across the entire temperature range in a manner that maintains the integrity of the battery material samples and the form factors housing such samples and allowing for other analysis, such as electrochemical analysis (ECA) and evolved gas analysis (EGA) to be performed simultaneously with heat flow measurements.

### BRIEF DESCRIPTION OF THE DRAWINGS

Advantages of this invention may be better understood by referring to the following description in conjunction with the accompanying drawings, in which like numerals indicate like structural elements and features in the various figures. For clarity, not every element may be labeled in every figure. The drawings are not necessarily to scale, emphasis instead being placed upon illustrating the principles of the invention.

FIG. 1 is a cutaway front view of a thermal analyzer system in which embodiments of the present inventive concept may be practiced.

FIG. 2A is a perspective view of a coin cell reinforcement apparatus, in accordance with some embodiments.

FIG. 2B is an exploded view of the coin cell reinforcement apparatus of FIG. 2A.

FIG. 2C is a cross-sectional view of the coin cell reinforcement apparatus of FIGS. 2A and 2B annotated with gas flow arrows.

FIG. 3A is a perspective view of a coin cell reinforcement apparatus, in accordance with other embodiments.

FIG. 3B is an exploded view of the coin cell reinforcement apparatus of FIG. 3A.

FIG. 4A is a perspective view of an open coin cell container, in accordance with some embodiments.

FIG. 4B is an exploded view of the open coin cell container of FIG. 4A.

FIG. 4C is a cutaway front view of the open coin cell container of FIGS. 4A and 4B.

FIG. 5A is a perspective view of an edge grip open container, in accordance with some embodiments.

FIG. 5B is an exploded view of the edge grip open container of FIG. 5A.

FIG. 5C is a cutaway front view of the edge grip open container of FIGS. 5A and 5B.

FIG. 6A is a perspective view of a sealed container, in accordance with some embodiments.

FIG. 6B is an exploded view of the sealed container of FIG. 6A.

FIG. 6C is a cutaway front view of the sealed container of FIGS. 6A and 6B.

FIG. 7A is a perspective view of a custom coin cell, in accordance with some embodiments.

FIG. 7B is an exploded view of the custom coin cell of FIG. 7A.

FIG. 7C is a cutaway perspective view of the custom coin cell of FIGS. 7A and 7B

FIG. 7D is a cutaway front view of the custom coin cell of FIGS. 7A-7D.

FIG. 8A is a perspective view of an electrochemical analysis (ECA) system, in accordance with some embodiments.

FIG. 8B is another perspective view of the ECA system of FIG. 8A.

FIGS. 9-11 are cutaway perspective views of the ECA of FIG. 8A and 8B, in accordance with some embodiments.

FIGS. 12A-12D are views of a teacup spring ECA silver lid alignment assembly, in accordance with some embodiments.

FIGS. 13A-13F are perspective and top views of a hair wire ECA assembly in charge cycling and overcharging operation and DSC operation modes, respectively, in accordance with some embodiments.

FIG. 14A is a perspective view of an open container, in accordance with some embodiments.

FIG. 14B is an exploded view of the open container of FIG. 14A.

FIG. 15A is a perspective view of a sealed container, in accordance with some embodiments

FIG. 15B is a partial section, front view of the sealed container of FIG. 15A.

FIG. 15C is an exploded view of the sealed container of FIGS. 15A and 15B.

FIG. 16A is a view of an electrical post assembly for an ECA configuration, in accordance with some embodiments.

FIG. 16B is a closeup view of the electrical post assembly of FIG. 16A.

FIG. 16C is an exploded view of a cell capture container including the electrical post assembly of FIGS. 16A and 16B.

FIGS. 17-22 are graphs of experimental results produced by a thermal analyzer system, in accordance with some embodiments.

### DETAILED DESCRIPTION

Reference in the specification to an embodiment or example means that a particular feature, structure or characteristic described in connection with the embodiment or example is included in at least one embodiment or example of the teaching. References to a particular embodiment or example within the specification do not necessarily all refer to the same embodiment or example.

The present teaching will now be described in detail with reference to exemplary embodiments or examples thereof as shown in the accompanying drawings. While the present teaching is described in conjunction with various embodiments and examples, it is not intended that the present teaching be limited to such embodiments and examples. On the contrary, the present teaching encompasses various alternatives, modifications, and equivalents, as will be appreciated by those of skill in the art. Moreover, features illustrated or described for one embodiment or example may be combined with features for one or more other embodiments or examples. Those of ordinary skill having access to the teaching herein will recognize additional implementations, modifications, and embodiments, as well as other fields of use, which are within the scope of the present disclosure as described herein.

In brief overview, embodiments of the present inventive concept include a battery material holder assembly, also referred to as a reinforcement apparatus, capsule, housing, or containment device, that confines the battery material for safe use during thermal analysis and testing, even when the melting point of the separator between the cathode and anode is exceeded, while also allowing simultaneous ECA and EGA analyses to be performed. In some embodiments, the holder assembly is constructed and arranged to maintain the integrity of a coin cell during an experiment, for example, described in embodiments herein. The coin cell can be thermally assessed without the need for a cell teardown where the battery is disassembled to analyze its individual components or combinations thereof. In other embodiments, internal elements of the coin cell (excluding the coin cell housing) such as the cathode, anode, separator, electrolyte, etc. are placed in a container that functions as the battery case but also functions as a container to hold the contents together. In some embodiments, the battery material holder permits differential scanning calorimetry (DSC) to be performed on single layer, Li-ion batteries in 2032 coin cells (CCs), but not limited thereto. The DSC analysis would be performed from −90-600° C. at 1° C./min, and measure onset temperature and enthalpies of reactions, with particular interest in decomposition reactions of the battery material. The invention disclosed is capable of DSC measurements ranging from −90° C. to at least 600° C., heating rates from isothermal to above 20° C./min (including cooling) with optional simultaneous EGA and ECA testing. Samples include single or multi-layer, Li-ion batteries, other battery chemistries, or other sample types. In some embodiments, the battery material samples are housed in coin cell capsules or the like. As used herein, a “sample” may include a standalone material or may refer to a coin cell including such material. A container 110 as used and described herein can hold a sample, i.e., either a battery material or a coin cell itself having battery material.

Accordingly, embodiments of the present inventive concept permit multiple analysis methods to be performed on battery materials including, but not limited to thermal, e.g., DSC, TGA, or DSC-TGA, electrical, e.g., charge cycling, or overcharging, evolved gas analysis, e.g., FTIR, MS, GC-MS, or FTIR|GC-MS, and/or failure modes, e.g., shorting, nail piercing or penetration, or heating. Some or all of these multiple analysis methods may be performed simultaneously, for example: DSC with EGA to an FTIR and voltage monitoring of the battery material. Battery materials may include but not be limited to individual battery components, combinations of battery components up to full and half cells, or combinations of battery materials with inactive battery materials (e.g., full cell stacked on a gold film). Full or half cells can be single layer or multilayer. The battery materials can be at a given state of charge, lithiation, or delithiation. The battery materials can have other physical, chemical or electrical modifications. Some embodiments may include non-battery materials including samples typically measured in the analysis method, e.g., indium, gold, or polymer, or samples like blowing agents, explosive material, or high energy material.

In some embodiments, a calorimeter may accommodate a coin cell of varying dimensions, for example, a 20 mm coin cell having a 3.2 mm thickness (e.g., a 2032 coin cell) but not limited thereto. Here, the calorimeter has a sensor that is capable of making measurements using such coin cells. For example, a heat flux DSC with a constantan/chromel diffusion bonded sensor is capable of <50 μW transitions. In another embodiment, a mechanical sensor hold-down mechanism can remove glitches due to CTE mismatches.

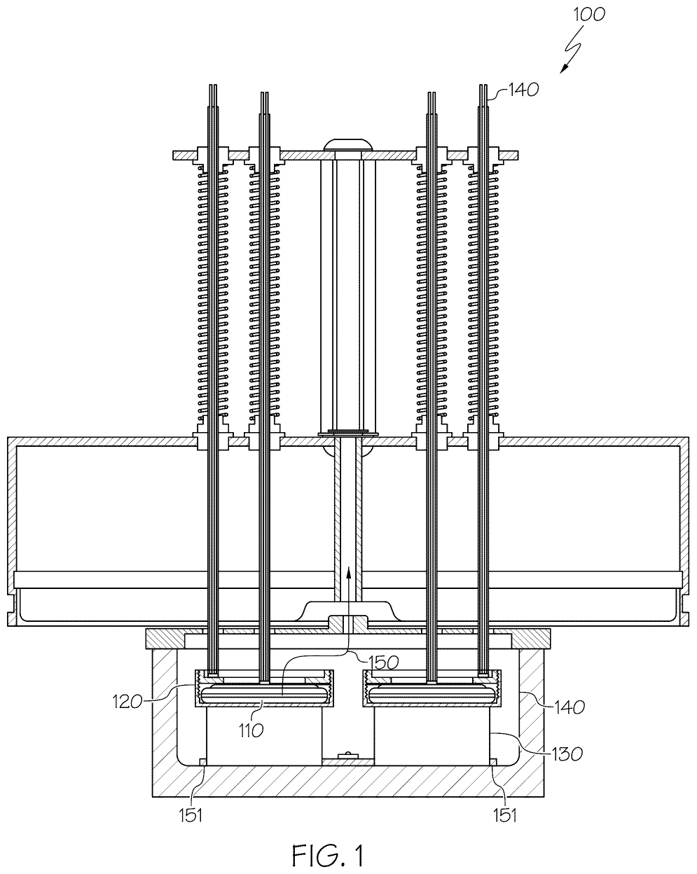

FIG. 1 is a cutaway front view of a thermal analysis system 100 in which embodiments of the present inventive concept may be practiced.

As shown in FIG. 1, the thermal analysis system 100, also referred to generally as an instrument 100 such as a calorimeter, can include a sample container 110, e.g., a DSC sample pan or the like, that contains a sample 120. The thermal analysis system also includes a thermal analyzer 130. In some embodiments, the thermal analyzer 130 is a DSC. In some embodiments, the sample 120 is a source battery material. In other embodiments, the sample is a coin cell which itself includes a battery material. Battery materials may be at a single layer coin cell scale, in full cell, half-cell, single component, or multicomponent combination. In other embodiments, samples may include non-battery material, such as indium for temperature calibration, air sensitive samples, or any material. In one example, sample 120 may be single layer, lithium-ion battery, housed in a 2032 coin cell case comprising a Lithium ion battery, for example, but not limited to a LiNiMnCoO₂ (622) cathode and carbon black anode with 100 μg 1:1 EC:DEC 1M LiPF₆ electrolyte solution with ˜4 mAh capacity at 100% state of charge. In another example, sample 120 may be a lithium-ion battery sample, e.g., sodium-ion, solid state electrolyte, or any other battery type.

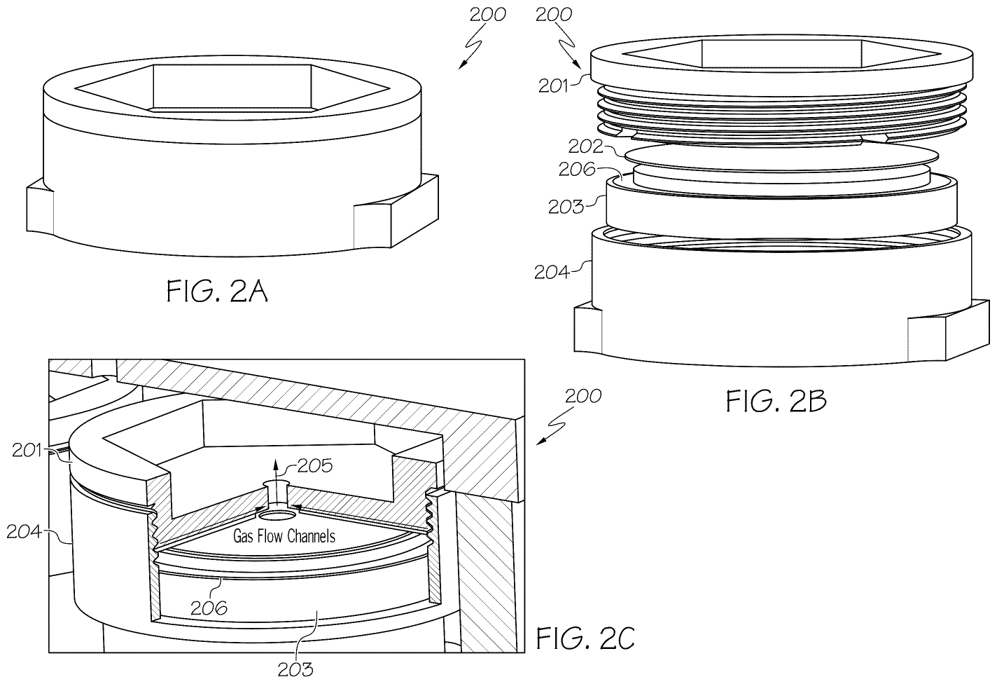

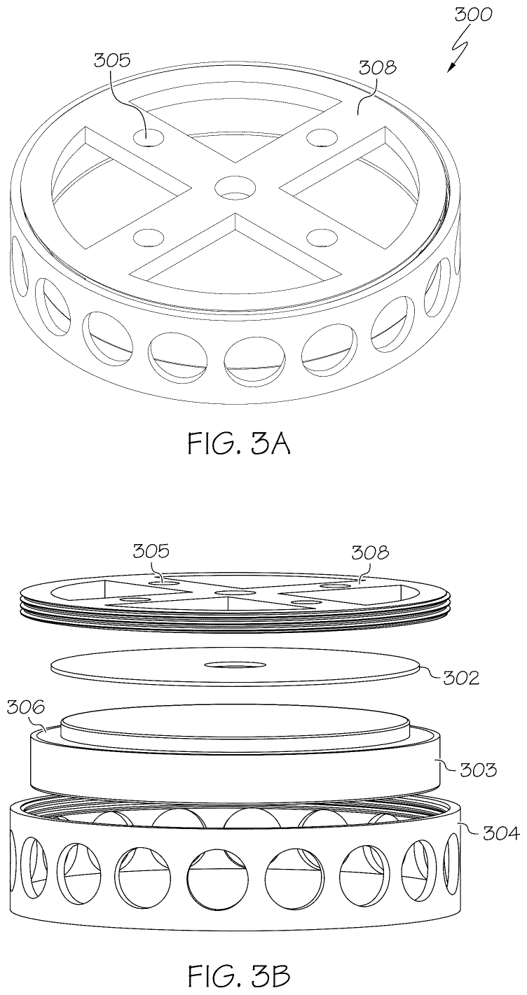

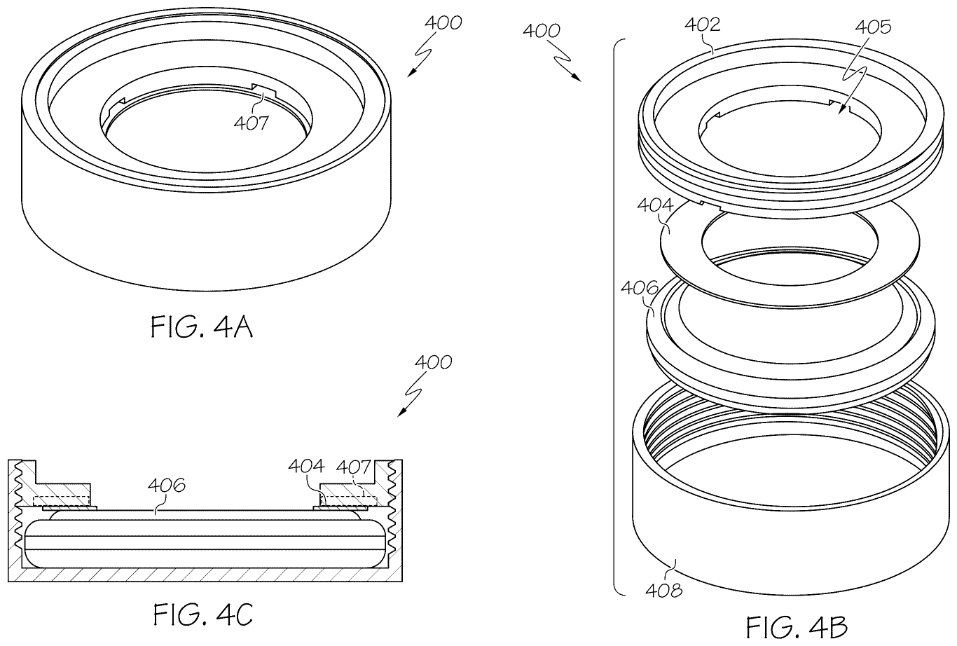

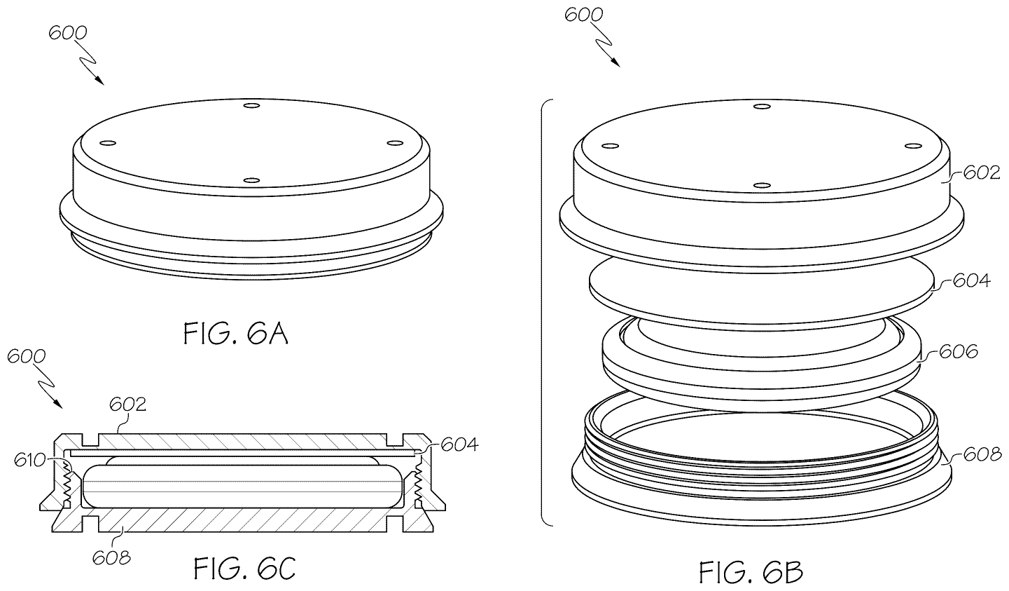

In some embodiments, the container 110 holds a battery material, for example, similar to or the same as the coin cell reinforcement apparatus 200 of FIGS. 2A-2C or apparatus 300 of FIGS. 3A-3B. In some embodiments, the container 110 is a sealed container, for example, shown and described with respect to FIGS. 6A-6C, which prevents any mass from entering or escaping the interior of the container 110 during an experiment. Any evolved gas generated will be contained in the sealed container. In other embodiments, the container 110 is an open container, for example, shown and described with respect to FIGS. 4A-4C, which allows mass to escape or enter during the experiment. Evolved gas generated during the experiment can be released from the container 110 and directed out of the thermal analyzer 130. The open container can be temporarily sealed allowing the container to be installed into the instrument 100 without contaminating or losing sample. Prior to an experiment start, the sealed container can then be opened, such as physically removing the seal. In some embodiments, the open container has a seal that can be removed through temperature changes, such as the coin cell's gasket melting, or through a ruptured burst disk at a given pressure differential. For either the sealed or open container option, the container 110 maintains thermal contact between sample of interest and the sensor throughout the temperature range.

In some embodiments, the instrument 100 can be connected to an EGA instrument including Fourier Transform Infrared Spectrometer (FTIR), Mass Spectrometer (MS), Gas Chromatogram with an MS detector (GC-MS), or any other EGA instrument. In the EGA configuration, an open or a venting pan is used, for example, described in embodiments herein.

In test environments where a complete battery is at a given state of charge (SoC) or during charge cycling, where the SoC is dynamically changing, the container 110 prevents unplanned external shorts between the cathode and anode during the experiment. In at least some embodiments herein, the container configurations described herein may allow an external short to be intentionally induced for the purpose of capturing measurements. The container 110 does not prevent internal shorts between the cathode and anode during the experiment. In most cases, an internal short is expected as a result of the experimental conditions, e.g., during a DSC experiment, the separator (typically formed of a polymer with a lower melting point than the stainless steel components of the pan) melts during heating of the container. An internal short circuit may occur when the positive and negative components of the battery connect directly due to the melted separator. This leads to rapid discharge of the battery, which can generate excessive heat. When a short circuit occurs in a battery, it can result in heat generation due to the rapid flow of current and the internal resistance of the battery. The instrument 100 can be used to measure this heat caused by the internal short. In particular, the instrument 100 may include temperature sensors to detect the heat release. This could provide insights into the energy dissipation of the battery, as well as potentially reveal the severity of the short circuit's effects. The thermal analysis system 100 may also include a set of option connectors 140 including a wiring assembly for monitoring voltage or other electrical characteristics of a sample in the container 110, and provides a conductive path for ECA or the like. The system 100 can therefore capture electrical measurements such as voltage, current, impedance, or others.

The thermal analysis system 100 also includes a path 150 for permitting evolved gas, for example, due to temperature and/or pressure changes, to exit the instrument 100 for analysis, for example, EGA, mass spectrometer, and so on. In addition, the thermal analysis system 100 allows charging and discharging the battery material 120 to age, and can monitor parasitic heat flows, or set a state-of-charge.

The thermal analysis system 100 can operate in a temperature Range from sub-ambient to >600° C., with a nominal temperature range −90-600° C. The heating rate may range between isothermal and greater than 20° C./min, with a nominal heating rate range between 0.5° C./min and 2.0° C./min. The instrument 100 can measure onset temperature and associated enthalpies from single layer (˜4 mAh) Li-ion battery decomposition reactions in the abovementioned temperature range.

The thermal analysis system 100 can be configured for several different operation modes such as DSC, overcharging, and charge cycling operation modes. In the DSC mode, an open or sealed container described in embodiments here may be used. Also, a simultaneous ECA may be performed that includes nominally voltage monitoring only to verify that the sample does not short internally.

In the overcharging mode, isothermal operation may be used, and an open or sealed container described in embodiments here may be used. The ECA 4-wire assembly 140 is required. Also, a simultaneous EGA testing may be performed that includes nominally mass spectrometry (MS) Fourier transform infrared (FTIR), or Gas chromatography-mass spectrometry (GC-MS), or any other EGA instrument. The instrument 100 includes relevant interfaces for coupling with such test systems.

In the charge cycling mode, isothermal operation may be used, and an open or sealed container described in embodiments here may be used, preferably a low mass, open container that allows electrical connections to the battery material. The ECA 4-wire assembly 140 is required. Also, a simultaneous EGA testing may be performed that includes nominally mass spectrometry (MS) Fourier transform infrared (FTIR), or Gas chromatography-mass spectrometry (GC-MS), or any other EGA instrument.

FIG. 2A is a perspective view of a coin cell reinforcement apparatus 200, in accordance with some embodiments. FIG. 2B is an exploded view of the coin cell reinforcement apparatus 200 of FIG. 2A. The coin cell reinforcement apparatus 200 is constructed arranged for use in the thermal analysis system 100 shown and described with reference to FIG. 1.

In some embodiments, the coin cell reinforcement apparatus 200 comprises a housing cap 201, an electrical insulator 202, and a housing case 204. The apparatus 200 can secure a coin cell 203 such as a 2032 coin cell but not limited thereto and therefore, other coin cells may apply equally. As shown, the entire coin cell, including its housing, gasket, and internal components such as cathode, anode, etc. is positioned between the housing cap 201 and case 204, which avoids the need for cell teardown that allows installation of battery components harvested from a complete battery into a sample container suitable for use in a conventional DSC.

The housing cap 201 (also referred to as a lid), and a housing case 204 (also referred to as a cup), when coupled together form a gas tight housing that contains the battery material 120 (FIG. 1) or coin cell 203 (FIG. 3). The gas tight housing seal can be configured to fail at a given pressure, temperature, or physical removal of the seal prior to an experiment performed by the thermal analysis system 100. In some embodiments, the battery material can be charged and discharged inside the housing, with the anode and cathode remaining electrically isolated at ambient and measurement conditions. Besides the gas tight housing seal, the housing containing the battery material does not yield or disassemble throughout the measurement. For calorimetric measurements, referring to FIG. 1, the housing maintains thermal conduction between the battery material 120 and the calorimetric sensor 151 throughout the measurement.

To achieve this, the housing cap 201 and case 204 of the coin cell reinforcement apparatus 200, also referred to as a coin cell capsule or holder, are threaded. In some embodiments, the insulator 202 is a mica insulator that provides electrical insulation between conductive components of the coin cell 203. In some embodiments, as shown in FIG. 2C, the housing cap 201 has at least one gas channel 205, or vent port. The coin cell reinforcement apparatus 200 reinforces the mechanical integrity of the 2032 coin cell during the measurement, preventing yielding or disassembly without shorting the battery. The gas channels 205 allow evolving gas to escape when the coin cell gasket 206 or O-ring or the like between the cathode and anode of the battery material predictively fails. In other embodiments, the coin cell capsule houses only the battery material, i.e., no coin cell housing, with gas being evolved a predictive internal pressure when a burst disk (described in other examples below) fails. In both cases, the capsule 200 maintains reasonable thermal contact between the battery material 120 (FIG. 1) or coin cell 203 (FIG. 2B) and the calorimetric sensor 151.

FIGS. 3A and 3B are views of another coin cell reinforcement apparatus 300, which is similar to the coin cell reinforcement apparatus 200 of FIGS. 2A-2C. However, the coin cell reinforcement apparatus 300 has a housing cap 301 and housing case 304 with a different construction. The housing cap 301 and housing case 304 and threaded for forming an expansion- resistant seal. However, the housing cap 301 has an x-shaped structure with additional gas channels 305. The two cross-members 308 forming the x-shaped structure permits the insulator 302 to be exposed and viewed from the top of the apparatus 303. In some embodiments, the housing case 304 has a plurality of additional channels 307 about its perimeter to reduce the heat capacity of the coin cell reinforcement apparatus.

FIG. 4A is a perspective view of an open coin cell container 400, in accordance with some embodiments. FIG. 4B is an exploded view of the open coin cell container 400 of FIG. 4A. FIG. 4C is a cutaway front view of the open coin cell container 400 of FIGS. 4A and 4B In some embodiments, the open coin cell container 400 is constructed and arranged for use in the instrument 100 shown and described with reference to FIG. 1.

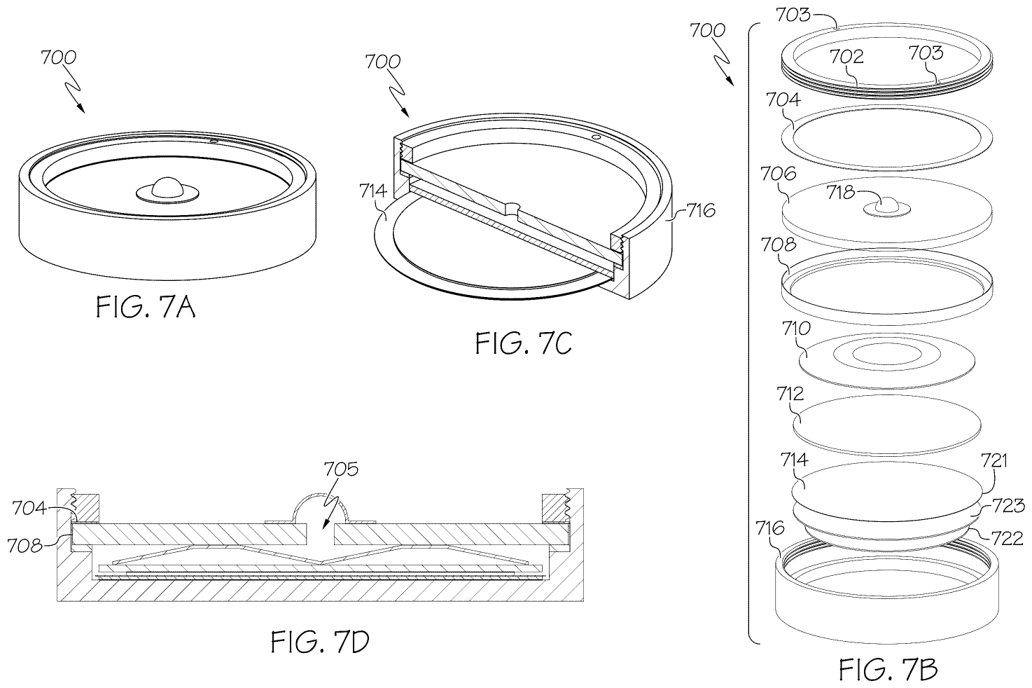

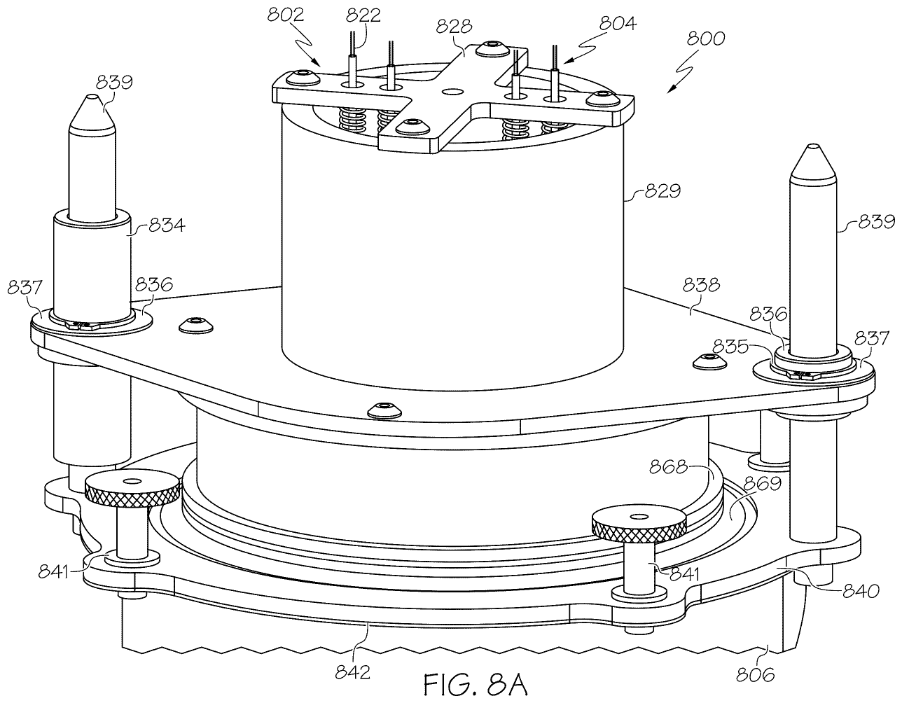

In some embodiments, open coin cell container 400 comprises a lid 402, a mica insulation disk 404, a battery material housing 406, and a cup 408. The lid 402 and cup 408 are structurally different than the housing cap 201, 301 and housing case 204, 304 of FIGS. 2A-3B, respectively, except that the cup 408 is constructed specifically for a container, while in other embodiments, for example, the custom coin cell in FIGS. 7A-7C has a cup that operates as a coin cell housing that houses a battery material, e.g., cathode, anode, and separator, without a container and can be used as a sample holder. The lid 402 and pan cup 408 thread together to clamp to the battery material housing 406. However, the lid 402 is for the open container and offers a wider vent port 405. The mica insulation disk is also shaped as a gasket and has a central hole that aligns with the vent port 405 to expose a top surface of the battery material housing 406. The open container 400 is also constructed and engineered to maintain a heat flow pathway between the battery material and a DSC sensor or the like while reducing errant thermal signals from deformation of the coin cell 406 holding the battery material during heating.

The container 400 rigidly holds the battery material housing 406, for example, a 2016-2032 coin cell, but not limited thereto, for thermal analysis and allows venting of gases from the coin cell 406. The coin cell 406 holding the battery is placed inside the cup 408, then the mica disk 404 placed on top, then the lid 402 is screwed down to contact the mica disk 404 and clamp the coin cell 406. The container 400 now containing the battery can be placed into a DSC or the like, for example, shown in FIG. 1, for thermal analysis. Optionally, battery components can be placed into the coin cell 406 for thermal analysis, e.g., cathode only, cathode and electrolyte only, etc.

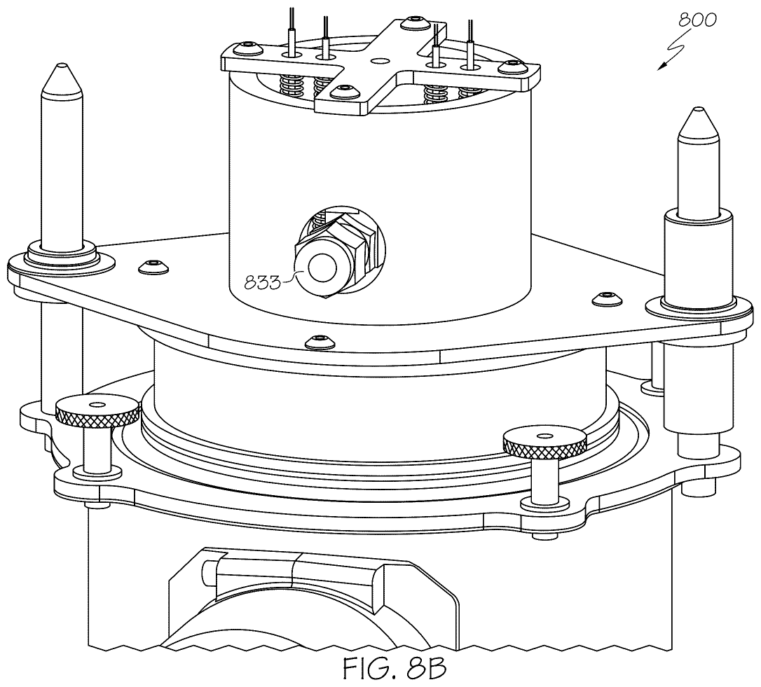

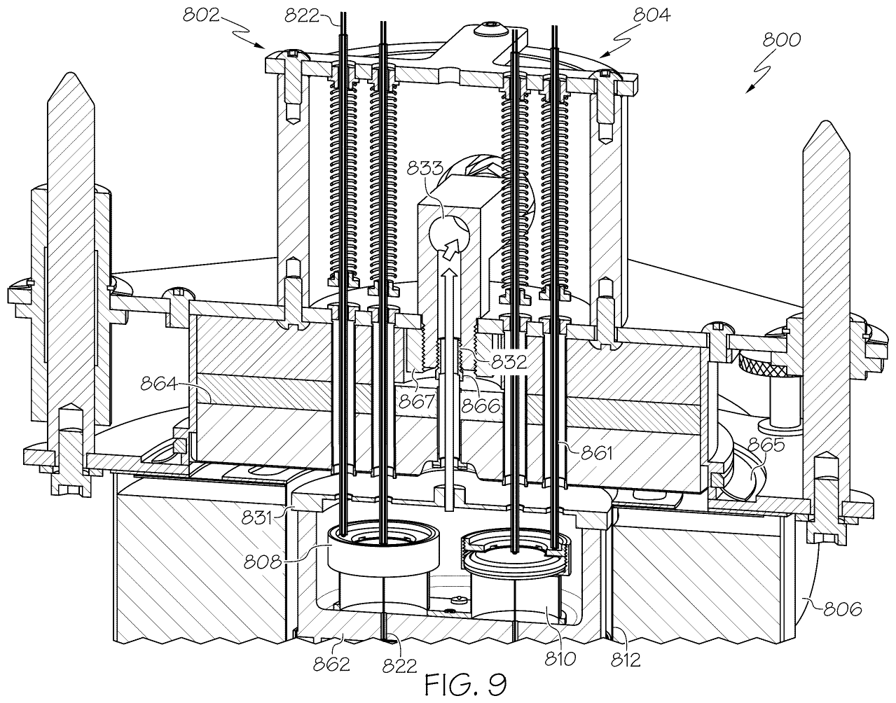

The container 400 can be scaled for larger or smaller coin cell form factors, pouch cells, cylindrical cells, or prismatic cells. The open container 400 allows for gases to evolve from the coin cell 406 during a thermal ramp. Evolved gas can then be analyzed using an FTIR, MS, etc. In some embodiments, the lid 402 includes at least one vent port 407 to allow for the escape of gasses during thermal and/or pressure-related testing, for example, when the cell gasket (not shown but similar to gasket 206 of FIG. 2B or gasket 306 of FIG. 3B) of the battery material in the cell housing 406. Container 400 facilitates ECA using e.g., the embodiments illustrated in FIGS. 8-11.

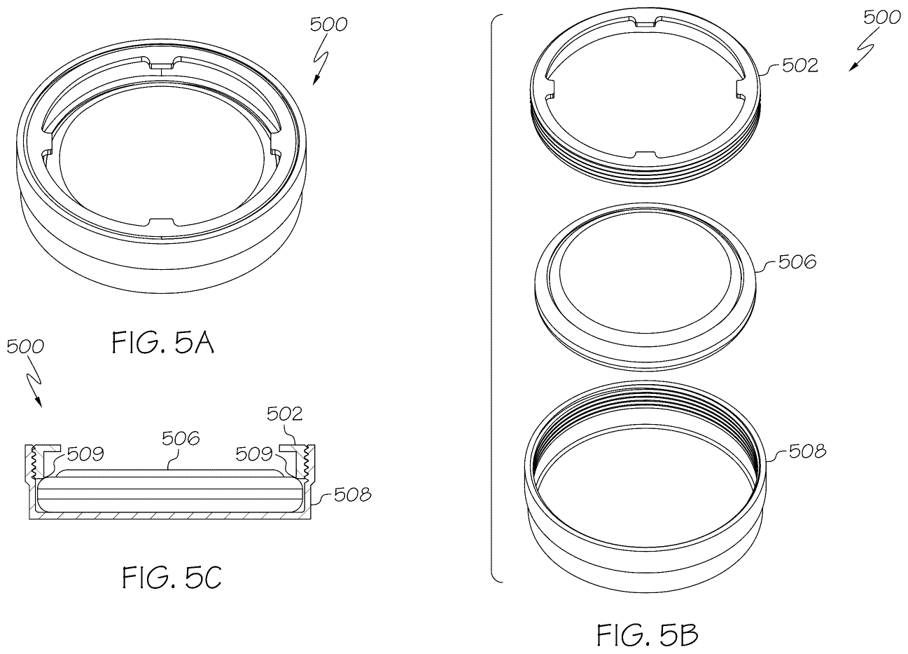

FIG. 5A is a perspective view of an edge grip open container 500, in accordance with some embodiments. FIG. 5B is an exploded view of the edge grip open container 500 of FIG. 5A. In some embodiments, the edge grip open container 500 is constructed and arranged for use in the instrument 100 shown and described with reference to FIG. 1.

In some embodiments, the edge grip open container 500 comprises a lid 502, and a low mass container cup 508, which may be similar to the pan cup 408 in FIG. 4A-4C, e.g., solid or sealed bottom region, etc. The container 500 can secure a battery material housing 506. The edge grip open container 500 does not have a mica insulator as with the container 400 of FIGS. 4A and 4B.

The edge grip open container 500 may be referred to as a low mass container edge crimp variant. Rather than crimping the coin cell from the top terminal, i.e., shown in FIG. 4C as a region including the mica insulation disk 404, the container 500 grips the coin cell 506 on edge of the bottom (e.g., positive or negative) terminal shown at region 509. The construction of the edge grip open container 500 further reduces the total mass of the battery material and containment, hence, further reduces the DSC time constant. Also, as previously mentioned, an additional insulator is not required.

FIG. 6A is a perspective view of a sealed container 600, in accordance with some embodiments. FIG. 6B is an exploded view of the sealed container 600 of FIG. 6A. FIG. 6C is a cutaway front view of the sealed container 600 of FIGS. 6A and 6B. In some embodiments, the sealed container 600 is constructed and arranged for use in the instrument 100 shown and described with reference to FIG. 1. The sealed container 600 can be used for accurate enthalpy measurements. In particular, accurate enthalpy measurements require constant mass. If mass changes during the experiment, e.g., decomposition of materials to evolved gas species, then accurate enthalpies cannot be determined.

In some embodiments, the sealed container 600 comprises a sealed container lid 602, an electrical insulator 604, and a seal container cup 608. The pan cup 608 may be similar to those in FIGS. 4A-5C, e.g., bottom surface unitary with sidewalls, etc. In some embodiments, the electrical insulator 604 includes a mica insulating material. In some embodiments, the pan cup 608 houses a battery material housing 606, for example, a coin cell type housing. The sealed container 600 also includes an interface 610 between the lid 602 and the cup 608.

Similar to an open capsule described in FIGS. 2A-5C, the sealed container 600 allows a battery material, for example, positioned in the coin cell housing 606, to be placed into the cup 608. The insulator 604 is positioned on the top of the coin cell housing 606 so that the coin cell 606 is sandwiched between the insulator 604 and the cup 608. The lid 602 has an interior thread that is screwed into the exterior thread of the cup 608. A special tightening tool may be used to apply an appropriate torque for forming a gas-tight seal between the lid 602 and the cup 608. container The sealed container 600 is not used for gas analysis experiments. However, analysis of the components post run is possible as all evolved components would be contained in the container. Since the container 600 is sealed when the lid 602 and cup 608 are coupled together, any gas species generated during the experiment would be contained inside the sealed container 600. A user could remove the container 600 post experiment, and analyze the internal components using a gas analyzer or other analytical methods.

FIG. 7A is a perspective view of a custom coin cell 700, in accordance with some embodiments. FIG. 7B is an exploded view of the custom coin cell 700 of FIG. 7A. FIG. 7C is a cutaway perspective view of the custom coin cell 700 of FIGS. 7A and 7B.

In summary, the custom coin cell 700 shown in FIGS. 7A-7D can include a construction that is similar to that of a 2032 coin cell or the like, with differences explained below that can prevent electrical shorts between the cathode 722 and anode 721 and allows charging, discharging, and aging of the battery material. The custom coin cell 700 can withstand a temperature range from ambient to 600° C. without yielding or disassembly. The custom coin cell 700 that can be used without a coin cell housing and allows venting at a given pressure. Using a burst disk and a mica seal, the polymer gasket typically used in coin cells is removed.

As shown in FIGS. 7A-7C, the custom coin cell 700 includes a closure ring 702 with one or more slots 703 for an installation tool, which can be used to thread the closure ring 702 against a disk cap 706 with a desired torque or other compression force. The disk cap 706 has a controlled vent and sealing disk cap having a vent port 705 (see FIG. 7C) and a burst disk 718 designed to vent at a given internal pressure. The custom coin cell 700 also includes a mica insulator 704 that electrically insulates the closure ring 702 from the cap 706, an isolating and compression seal ring 708 that is composed of mica or some other sealing material that is also electrically insulative, a spacer 712 commonly used in standard coin cells, a spring 710 that applies pressure from the cap 706 to the spacer 712, a battery material 714 nominally a single layer Li-ion cathode 722, separator 723, and anode 721, and a cup 716. During operation, the spring 710 applies pressure between the cap 706 and the spacer 712, which clamps and applies pressure to the battery material 714.

To assemble the custom coin cell 700, the desired battery material 714 is installed inside the cup 716, then the spacer 712 is placed on top the battery material 714 in the cup 716. The spring 710 is then installed on the spacer 712, followed by the compression seal ring 708, cap 706, and closure ring 702. The cap 706 is then pushed down till it compresses the compression seal ring 708 for adequate sealing and required pressure through the spring 710 and onto the battery material 714. The closure ring 702 is then screwed down, e.g., using an installation tool engaging with the slots 703, to capture the closure ring 702 and cap 706, maintain adequate sealing between cap 706 and compression seal ring 708, and prevent unintentional disassembly.

After the custom coin cell 700 is assembled, the cap 706 and cup 716 are electrically connected to the negative terminal and positive terminal, respectively, while maintaining electrical isolation between the two. Newly assembled battery materials can then be formed using a charge cycler or formed battery material can be set to a given state of charge (SoC).

The burst disk 718 can be constructed and arranged to vent at a given internal pressure. Alternatively, the vent port 705 located in the cap 706 can be covered with a manually removeable seal, e.g., tape, that would be removed prior to experimental start. The vent port 705 can be dimensioned with a small ID, like a pin hole, to provide venting throughout the experiment, leading to continual EGA time aligned with the thermal analysis.

Accordingly, the custom coin cell 700 can house coin cell sized samples without using a standard coin cell housing. Features may include reduced mass, no polymer gasket, easier to tear down, controlled vent pressure, and a pin hole option with no burst disk offered by the custom coin cell 700.

As shown in FIG. 7D, the capsule allows internal gas species to escape through flow paths to a single point or can have multiple points for the gas to escape. The gas release mechanism can remain sealed until a given condition is met. Examples may include using internal pressure to break a burst disk, using temperature to melt or weaken a seal that flows out of the gas path or internal pressure pushes the seal out or away from the gas path, and using seal that is physically removed or damaged by the user or mechanically like a piece of tape that is removed or pierced. The gas release mechanism can restrict gas flow. Examples include a gas flow path with a restricting mechanical design like a pin hole exit point or a membrane. The gas release mechanism can combine multiple options or a device can have multiple release mechanism. An example is a burst disk that covers a pin hole. When a certain pressure is reached, the burst disk fails and begins releasing gas. The pin hole then continues to restrict the gas that is released.

FIG. 8A is a perspective view of an electrochemical analysis (ECA) system 800, in accordance with some embodiments. FIG. 8B is another perspective view of the ECA system 800 of FIG. 8A. FIGS. 9-11 are cutaway perspective views of the ECA system 800 of FIG. 8A and 8B, in accordance with some embodiments.

In some embodiments, as shown, the electrochemical analysis (ECA) system 800 comprises a plurality of assemblies 802-812, in particular, a spring-loaded contact ECA lid assembly 802, an electrical wire assembly 804, a cooler assembly 806, a container assembly 808, a coin cell assembly 810, and a DSC cell assembly 812.

The spring-loaded contact ECA system 800 Assembly 802 houses one or more, preferably four, electrical wire assemblies 804A-804D (generally, 804). In some embodiment, an electrical wire assembly 804 each comprises a quartz insulating tube 821 and at least one electrical wire 822 or related conductor. As shown in electrical wire assemblies 804A and 804B, electrical contact is made to a positive terminal and negative terminal on the sample, respectively. As shown in FIG. 9, ends of the wire are exposed outside the DSC cell assembly 812. Each electrical wire assembly 804A and 804B has two separate wires exposed: one for a current carrying connection and one for a voltage sensing connection of the desired terminal polarity. This configuration allows a four-terminal sensing, or 4-wire connection, for charge cycling or overcharging ECA, and are used for a charge cycling and overcharging operation mode. For a DSC operation mode, the 2-wire interface for voltage monitoring can be used either or both wires from a single electrical wire assembly 804 to perform the measurement. The other electrical wire assemblies 804C and 804D contact the thermally and electrically inactive reference container, but only serves to preserve thermal symmetry required for DSC measurements.

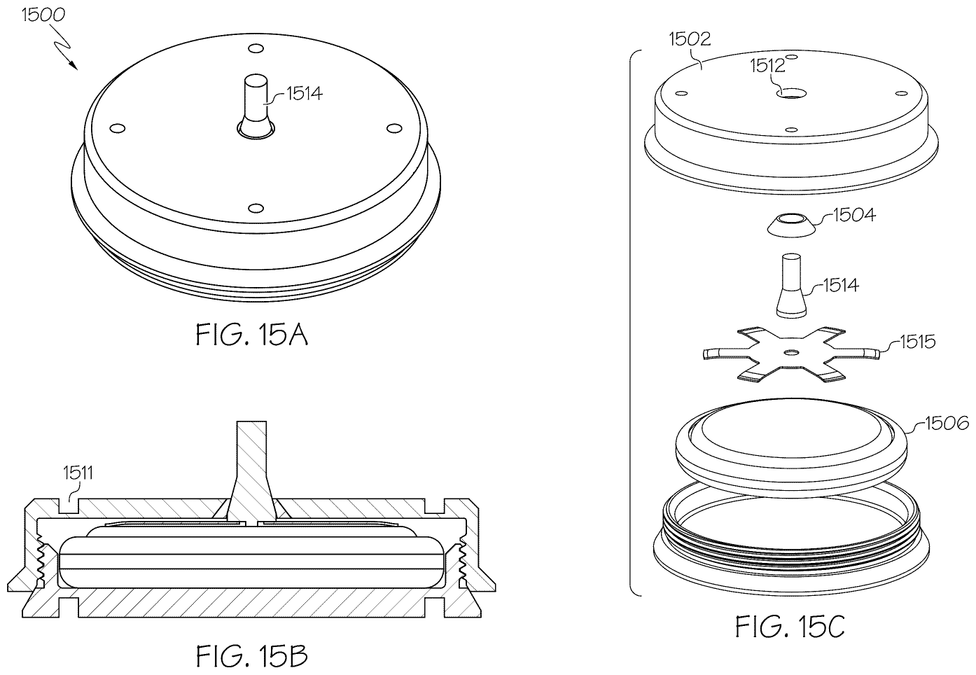

In some embodiments, the pan assembly 808 is depicted as an open container design, for example, similar to or the same as open containers of FIGS. 4A-4C. In other embodiments, the edge grip open container shown and described with reference to FIGS. 5A-5C can also be used with the spring-loaded contact ECA system 800. In other embodiments, the sealed container edge seal of FIGS. 15A-15C is also compatible if using the ECA pin modification. In other embodiments, The custom coin cell with burst disk 700 of FIGS. 7A-7C is also compatible with the spring-loaded contact ECA system 800 if the alignment of the burst disk and electrical wire assembly 804 do not interfere. Such an interference would compromise the ECA, burst disk operation, or both. Other container designs are also compatible with the teacup spring ECA system 800 to accommodate alternative CC form factors, custom form factors, and other.

While using an open container, for example, FIGS. 4A-5C, the spring-loaded contact ECA system 800 has an EGA gas flow path (shown by flow arrows) that allows evolved gas escaping the pan assembly 808 to be directed out of the DSC cell assembly 812 via an opening in the silver lid 831 for the DSC cell assembly 812, through the spring-loaded contact ECA lid assembly 802 via the gas flow tube 832, and into and out of the EGA port interface 833, or standard type fitting. The EGA port interface 833 can easily accommodate ¼″ OD tubing, but not limited thereto to be plumbed to an EGA system, e.g., FTIR, MS, GC-MS, or others.

An alignment of the spring-loaded contact ECA lid assembly 802 to allow ECA using the electrical wire assemblies 804 is accomplished using a set of alignment features in both the teacup spring ECA lid assembly 802 and the cooler assembly 806. In some embodiments, a long alignment bushing 834 and short alignment bushing 835 are attached to the spring-loaded contact ECA lid assembly 802 using clamps 836 and washers 837 to a mount plate 838. These bushings 834, 835 can interface to alignment posts 839 on the cooler assembly's alignment feature mount plate 840. The alignment posts 839 and long alignment bushing 834 are first interfaced, and the spring-loaded ECA lid assembly 802 is then slowly lowered into place. As it is lowered, the short alignment bushing 835 is then rotated into position. Tapering at the tops of the alignment posts 839 facilitate installation of the assembly. Initial installation and alignment of the assembly's alignment feature mount plate 840 to the cooler assembly 806 is accomplished by a thumbscrews 841 locking the mount plate 840 to the cooler head interface plate 842.

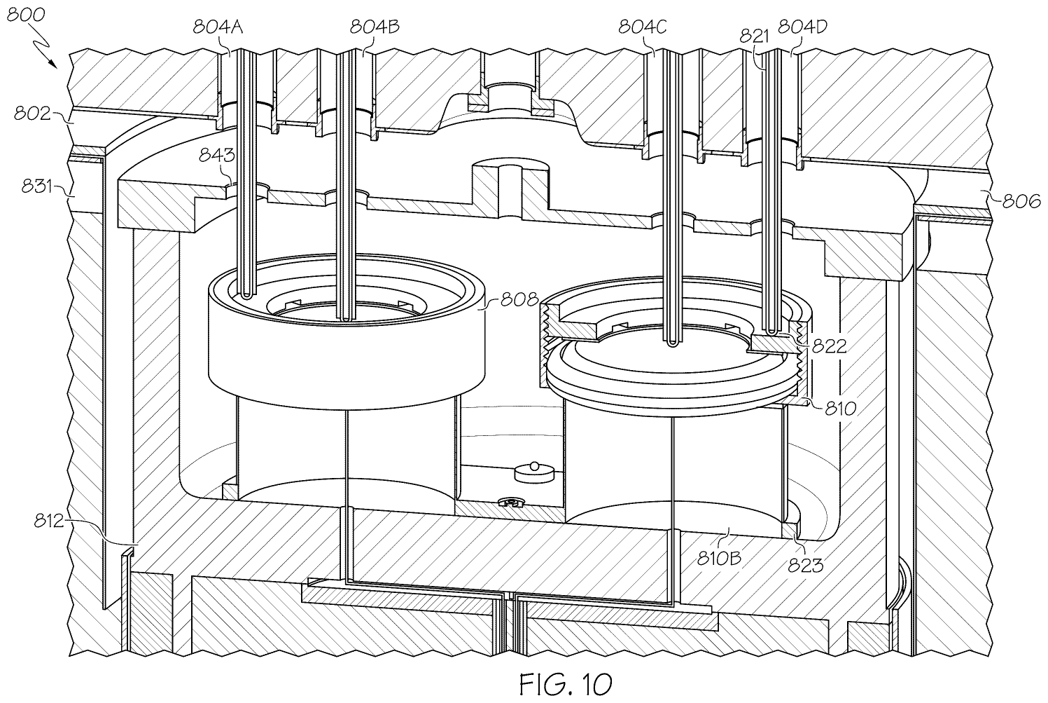

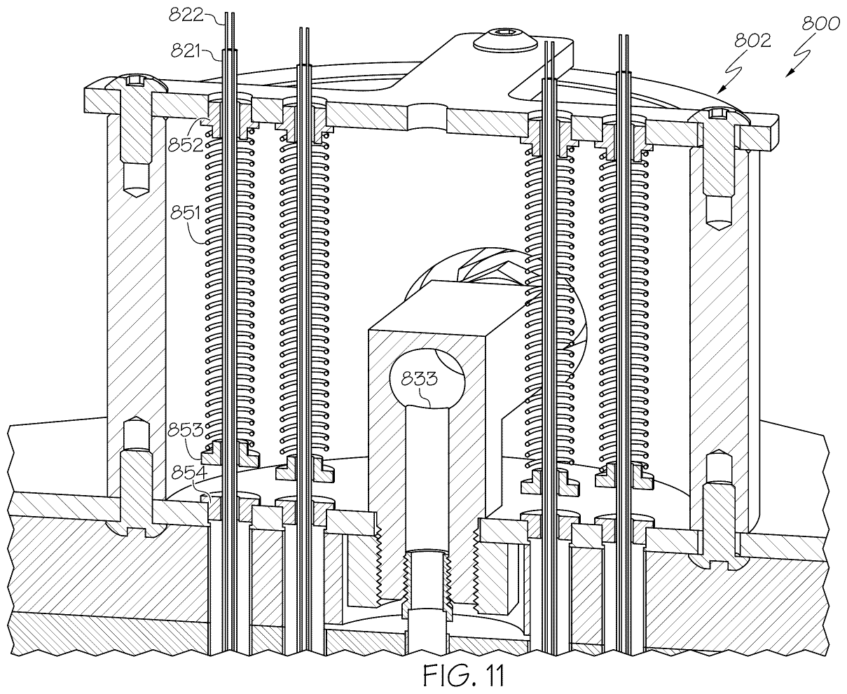

To provide adequate contact between the electrical wire assemblies 804 and pan assembly 808, as shown in FIG. 11, springs 851 are used that connect to fixed guide and spring locking bushings 852 and quartz-to-spring interface bushings 853. When the spring-loaded contact ECA lid assembly 802 is installed, lowering of the assembly 802 pushes the electrical wire assemblies 804 up, thereby compressing the springs between the two bushings. The fixed guide and spring locking bushings 852, along with fixed guide and plate mounted bushings 854 can guide the electrical wire assemblies 804 to maintain proper positioning and keep the assemblies 804 concentric to the electrical wire assembly passthrough tubes 861 and silver lid 831 for through holes 843 in the silver lid 831 (see FIG. 10). The fixed guide and plate mounted bushings 854 are installed into the mount plate 838. The mount plate 838 also has installed a cylindrical support 829, which interfaces to the fixed guide and spring locking bushings 852 via a cross-shaped interface 828.

The DSC cell assembly 812 is shown with a DSC silver cell block 862, silver lid for DSC cell 831, and DSC sensor 823. The DSC Cell Assembly 812 has been dimensionally designed to accommodate heat flow measurements for the pan assemblies 808. Similarly, the cooler assembly 806 was dimensionally designed for the pan assemblies 808 and to accommodate the DSC cell assembly 812 both dimensionally and for temperature range (sub-ambient->600° C.).

Thermal insulation 864 of the teacup spring ECA lid Assembly 802 is held in place by a lid bottom plate 865. The plate 865 also interfaces the passthrough tubes 861 and gas flow tube 832. The gas flow tube 832 also interfaces a gas flow tube adapter screw 866, and the gas flow tube 832 in turn interfaces the EGA port interface 833 (see FIG. 8B). A EGA interface adapter screw 867 clamps the EGA port interface 833 to the teacup mount plate 838. The different passthrough tubes 832 and 861 and insulation 864 are clamped between the mount plate 838 and lid bottom plate 865, and held at a distance by an insulator can 868, which also serves to house the insulation 864. To prevent outside air from impinging the DSC cell assembly 812 or EGA flow path (see flow arrows), a sealing grommet 869 encompasses the bottom of the insulator can 868. In some embodiments, the interface, or EGA port, has a connector for coupling to an FTIR, mass spectrometer, or other instrument.

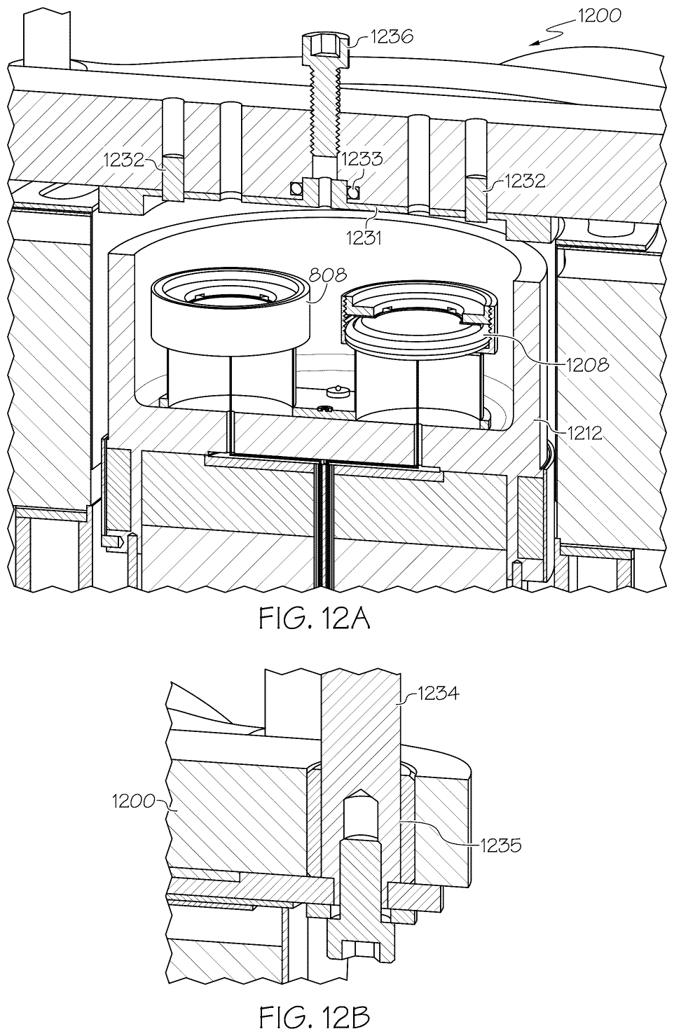

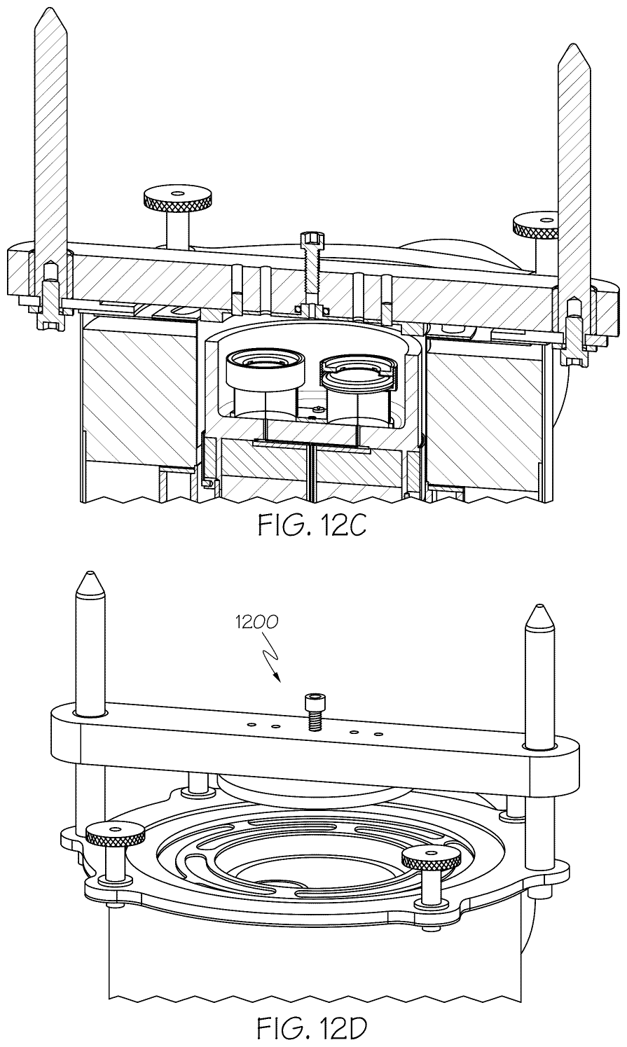

FIG. 12 is a cutaway perspective view of a spring-loaded contact ECA silver lid alignment assembly 1200, in accordance with some embodiments. In describing the silver lid alignment assembly, reference is made to FIGS. 8A-11. At some elements of FIG. 12, for example, coin cell assemblies 1210 may be similar or the same as counterpart elements of FIGS. 8A-11, for example, coin cell assemblies 810.

Referring to FIGS. 12A-12D, to accommodate the teacup spring ECA system 800, the silver lid 1231, which may be similar to or the same as the silver lid 831 of FIG. 9, first needs to be properly aligned. In some embodiments, the alignment posts 839 in FIG. 8A are aligned to the sensor. Once the sample and reference coin cell assemblies 1210 have been installed, the silver lid 1231 is ready for installation. Accomplishment of alignment of the silver lid 1231 is facilitated by the silver lid alignment assembly 1200. The alignment assembly 1200 first holds the silver lid 1231 in place using alignment pins 1232 to position the lid 1231 and a holding O- ring 1233. After the silver lid 1231 is attached, the alignment assembly 1200 can be lowered over the alignment posts 1234, which interface via alignment bushings 1235 on the alignment assembly 1200. After the lid 1231 and alignment assembly 1200 is installed, a detachment screw 1236 is used to push the lid away from the assembly 1200 and into place in the DSC cell assembly 1212. The alignment assembly 1200, now with lid 1231 detached, can be removed from the alignment posts 1234.

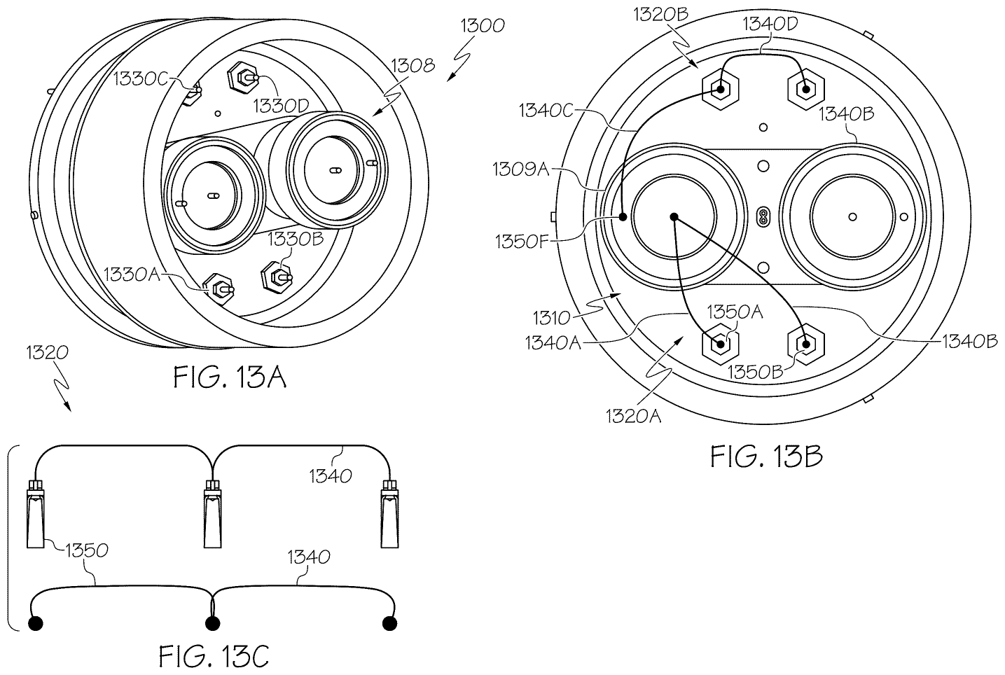

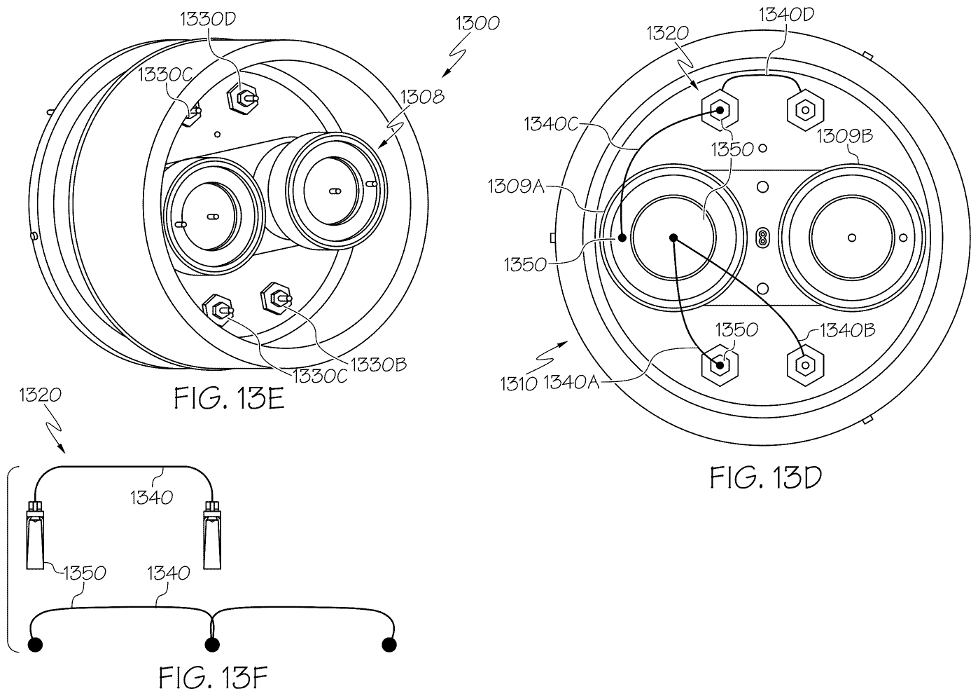

FIGS. 13A-13F are perspective and top views of a hair wire ECA assembly 1300 in charge cycling and overcharging operation and DSC operation modes, respectively, in accordance with some embodiments.

The hair wire ECA assembly 1300 allows simultaneous ECA measurement with thermal analysis of a sample, while allowing EGA if desired. ECA is necessary for the operation modes: overcharging and charge cycling (shown in FIGS. 13A-13B) and can be used in the DSC operation mode (shown in FIGS. 13C-13F).

In some embodiments, the hair wire ECA assembly 1300 comprises an open container with pin assembly 1308 comprising a sample pan 1309A and a reference pan 1309B, a DSC cell assembly 1310, a hair wire assembly 1320, a set of pin feedthrough assemblies 1330, a hair wire assembly 1340, and a hair wire pin connector assembly 1350.

In some embodiments, the pin feedthrough assemblies 1330 comprise four pin feedthroughs located at the base of the DSC silver cell block, two sets of hair wire assemblies 1320, and a sample pan assembly 1308 with pin connectors. The pin feedthrough assembly 1330 can include plurality of pin feed through connectors 1330A-1330D, for example,a current carrying positive (+) connector, a voltage sensing positive (+) connector, a current carrying negative (−) connector, and a voltage sensing negative (−) connector. The hair wire assembly 1340 comprises a plurality of hair wires 1340A-1340D, for example, a hair wire current carrying to sample positive (+) terminal, a hair wire current sensing to sample positive (+) terminal, a hair wire current carrying to sample negative (−) terminal, and a hair wire current sensing to sample negative (−) terminal. For the overcharging and charge cycling operation modes, a four-terminal sensing, or four-wire connection is used. In the four-wire connection, the hair wire assembly 1320 can comprise 3 pins and 2 hair wires, connected in parallel, provide electrical contact between a sample terminal, either (+) or (−), to the feedthrough pins' current carrying pin and voltage sensing pin for the given polarity.

The DSC operation mode only monitors voltage of the battery throughout the temperature range; hence, a four-wire connection is not necessary as only two-wires are required. The hair wire assembly 1320 for the DSC operation mode consists of only two pins and one hair wire. The hair wire assembly 1320 in the DSC operation provides electrical contact between the sample terminals (positive and negative) and various voltage sensing pin connectors, for example, as shown.

For quality thermal analysis using a differential measurement in any operation mode, a reference pan 1309B is used with similar thermal properties as the sample pan 1309A but without any hair wire assembly installed to the reference pan 1309B.

The hair wire and pins are constructed and arranged to provide electrical contact between the pins in the bottom of a DSC cell, or more specifically, pin feedthrough assemblies 1330A-1330D and the pins on the capsules, e.g., described in embodiments herein. The pins are to provide reasonable electrical contact across the temperature range: DSC Operation Mode: sub-ambient->600° C.; Charge Cycle and Overcharging Operation Mode: sub-ambient->150° C. In some embodiments, alternative pans 1309 can be used with appropriate (+) and (−) terminal connectors. The hair wire ECA also allows (+) and (−) terminals for batteries to be flipped, or formulated in reverse. The hair wire ECA can also flip current carrying and voltage sensing feedthroughs for similar terminal polarity if desired for either a first option, i.e., connect at the sample pin 1309A or the second option, i.e., connect at a feedthrough pin.

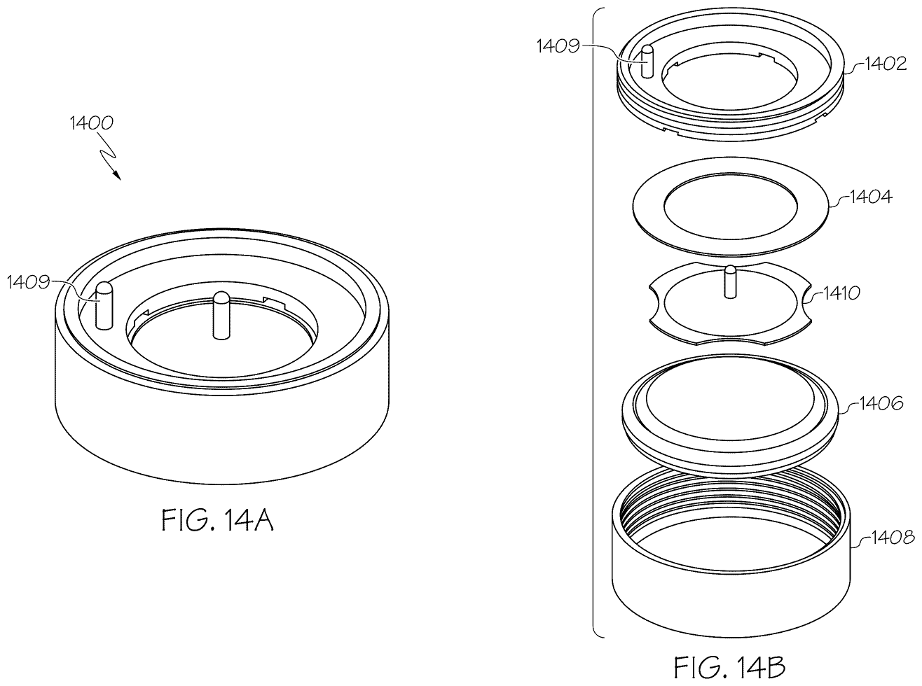

FIG. 14A is a perspective view of an open container 1400, in accordance with some embodiments. FIG. 14B is an exploded view of the open container 1400 of FIG. 14A. The open container 1400 may be similar to the open container 400 pin lid of FIGS. 4A-4C, except that the lid 1402 has a first pin 1409, and the open container 1400 also has a low mass capsule chip pin 1410.

In some embodiments, the open container 1400 can be equipped with pin 1409, and capsule chip pin 1410 to accommodate the hair wire ECA configuration shown and described in FIGS. 13A-13F. Components are identical to the open container 400 described except for the lid 1402 which has a pin terminal 1409. To accommodate the other polarity, a low mass capsule chip pin 1410 is installed that is positioned between the mica insulation disk 1404 and battery material housing 1406. Acceptable form factors of the battery 1406 and sample types are the same for both the open container 400 and open container with pins 1400 in FIG. 14.

FIG. 15A is a perspective view of a sealed container 1500, in accordance with some embodiments. FIG. 15B is a front partial section view of the sealed container 1500 of FIG. 15A. FIG. 15C is an exploded view of the sealed container 1500 of FIGS. 15A and 15B.

The sealed container 1500 can be equipped with pins to accommodate the hair wire ECA configuration shown and described in FIGS. 13A-13F. Components are similar to or identical to the sealed container described in FIGS. 6A-6C except that the lid 1502 has an insulator or seal 1504 and a feedthrough port 1512 to accept a pin 1514. The pin 1514 to electrically contact the battery top, interfaces the battery with a spring electrical contact 1515, also referred to as an electrical feedthrough spring, that is affixed to the pin 1514. An insulator 1504 sits between the pin 1514 and lid 1502, providing electrical isolation from the pin 1514 to the lid 1502, and, hence, the battery terminal on the bottom. The other polarity electrical connection can be installed into one of the four recess points 1511 in the lid 1502. The insulator 1504 also seals the pin 1514 to the lid 1502. Acceptable battery form factors and sample types are the same for both the sealed container and sealed pan with pins. Acceptable form factors of the battery 1506 and sample types are the same for both the sealed container 600 of FIGS. 6A-6c and sealed container 1500 with pin 1514.

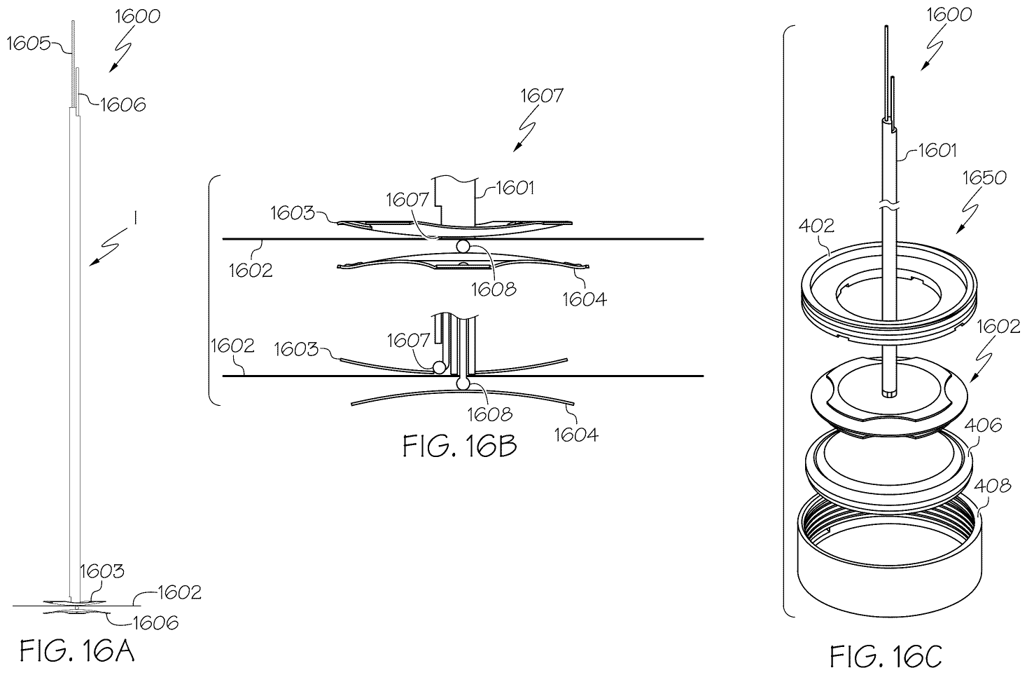

FIG. 16A is a view of an electrical post assembly 1600 for an ECA configuration, in accordance with some embodiments. The electrical post assembly is an alternate embodiment for performing ECA to the spring-loaded contact ECA assembly shown in FIGS. 8-12. FIG. 16B is a closeup view of the electrical post assembly 1600 of FIG. 16A. FIG. 16C is an exploded view of a cell capture container 1650 including the electrical post assembly 1600 of FIGS. 16A and 16B.

In some embodiments, the electrical post assembly 1600 comprises a quartz insulation tube 1601, an electrical insulation disk 1602, a positive chip spring 1603, a negative chip spring 1604, a positive wire 1605, a negative wire 1606, a positive wire weld 1607, and a negative wire weld 1608. In some embodiments, the electrical post assembly 1600 can be assembled with the open coin cell container 400 of FIGS. 4A-4C, but not limited thereto.

In FIGS. 16A-16C, the electrical wires 1605, 1606 are connected to a battery 1616, by chip springs clamped by the open container's lid 402. The electrical post assembly 1600 comprises a positive wire 1605 and negative wire 1606 that extend through the quartz insulation tube 1601. The base of the wires 1605, 1606 are welded to chip springs 1603, 1604 at the positive wire weld 1607 and negative wire weld 1609, respectively, which provide electrical contact between the wires 1605, 1606 and springs 1603, 1604. An electrical insulation disk 1602, which can be mica or other electrically insulative material, separates the two opposite polarity chip springs 1603, 1604 from each other. When the lid 402 is installed, the force compresses the two chip springs 1603, 1604 facilitating electric contact with the respective battery terminals, i.e., of the battery material 406. In use, electrical post assemblies are installed in both sample and reference coin cell capsules to preserve thermal symmetry necessary to satisfy symmetry of sample and reference measuring systems required in practicing DSC.

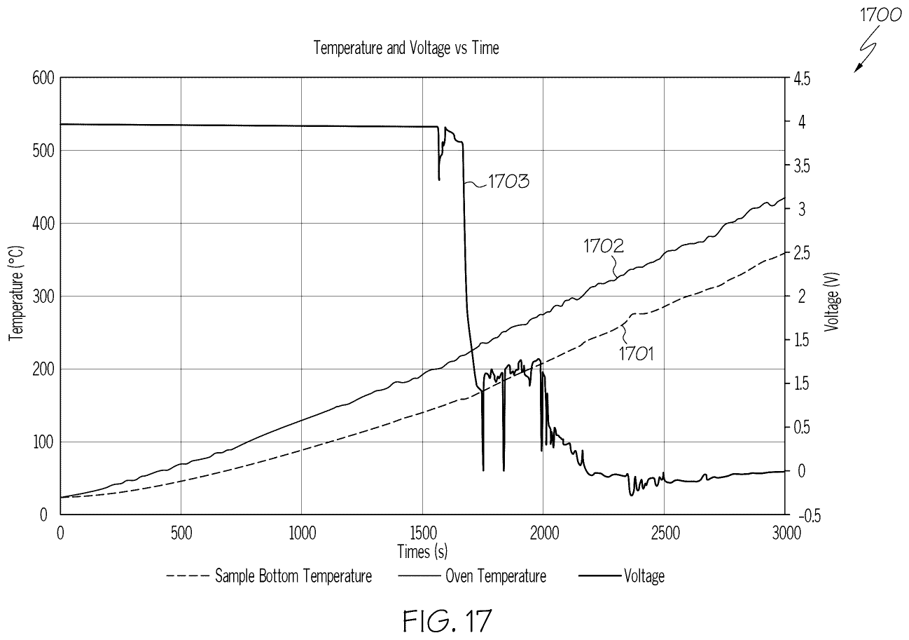

FIG. 17 illustrates voltage monitoring results produced using the coin cell reinforcement apparatus 200 of FIGS. 2A-2C positioned in the thermal analysis system 100 of FIG. 1, which performs a voltage monitoring experiment during a temperature ramp and a DSC-EGA-FTIR experiment. Both experiments were conducted from temperatures ranging from ambient to 600° C. at temperature ramps from 0.1-5° C./min. As shown in the graph 1700 of FIG. 17, which is performed using the coin cell reinforcement apparatus 200 of FIGS. 2A-2C, a voltage plot 1703 across a temperature range 0-600° C. is shown to drop at 1500 seconds as compared to temperature plots of a sample bottom temperature 1701 and oven temperature 1702, respectively.

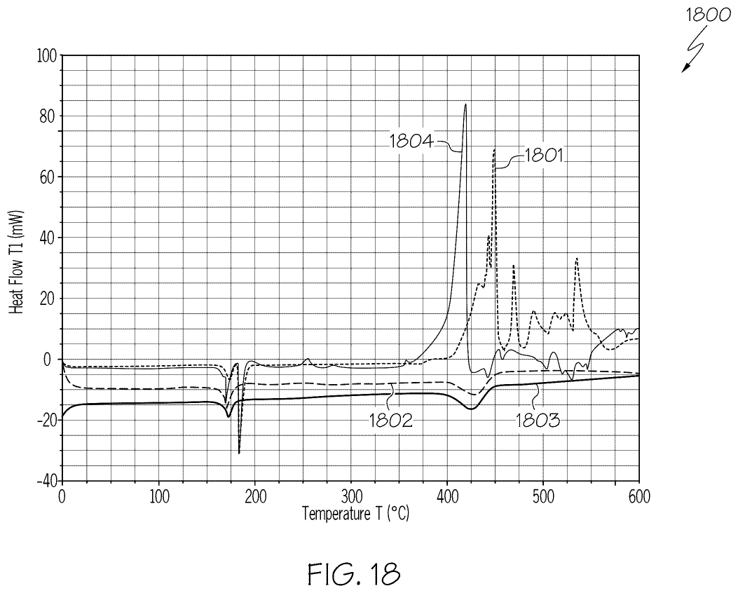

FIG. 18 illustrates a graph 1800 of measurements during testing of battery component materials. The thermal analyzer in accordance with embodiments of the present inventive concept, e.g., described above, can perform such measurements. During these experiments, the conditions include a heating rate of 1° C./minute and an N2 purge.

As shown, a DSC experiment with an empty coin cell coin cell case with a polypropylene gasket (1803) shows melting and subsequent decomposition of the gasket. 1802 shows DSC experiment of LCHE electrolyte in a sealed coin cell case. 1801 shows a DSC experiment of Li in a sealed coin cell case, and 1804 shows a DSC experiment of Li and LCHE electrolyte in a sealed coin cell case. The combination of this data(1801-1804) demonstrates the reduction in reaction onset temperature as a result of combining Li with LHCE electrolyte in a sealed coin cell. In this way, the process allows for the thermal analyzer to be used to understand various reaction mechanisms.

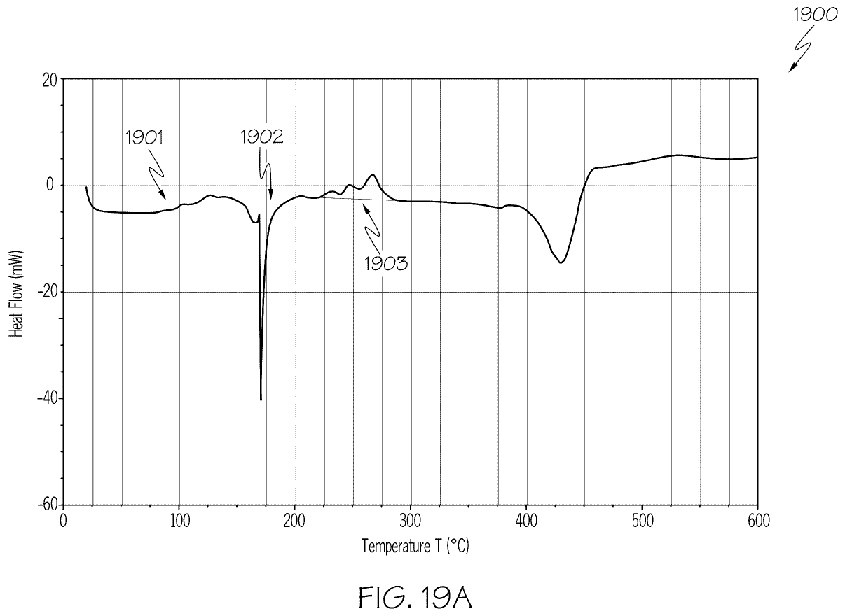

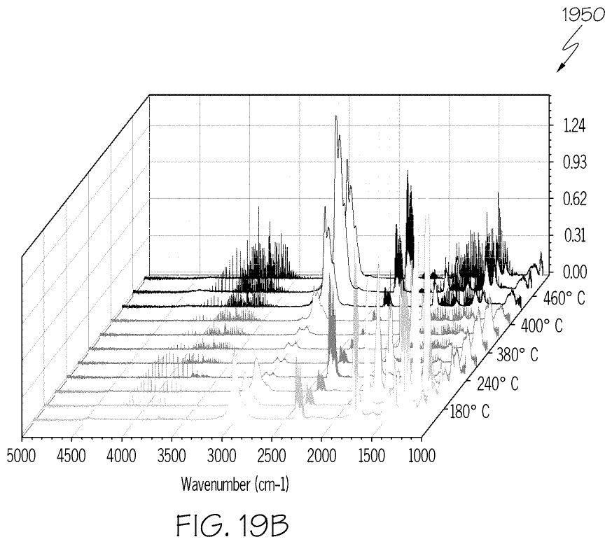

FIG. 19A is a graph 1900 illustrating measurements related to identifying reaction onset temperatures and total energy performed by a thermal analyzer in accordance with some embodiments. As the coin cell is heated, the temperature increases. During this experiment, the conditions included a heating rate of 1° C./minute and an N2 purge. Plot 1901 is at a first region of the graph 1900 where a cell degradation onset temperature or SEI reorganization and decomposition of the coin cell occurs. The lid and cup of the coin cell under test, e.g., a nickel manganese cobalt (NMC) 532 coin cell but not limited thereto, begin to separate as the sealing gasket, typically polypropylene, melts, for example, around between 160-175° C. The gasket begins to decompose at about 400° C. During the melt and decomposition, evolved gas can escape the cell and be analyzed using an evolved gas analyzer, such as an FTIR. Plot 1902 is at a second region of the graph 1900 that illustrates a reaction onset temperature and shows the gasket melting and subsequent venting of gaseous products, for example, where the coin cell gasket and separate experience a melting point due to the temperature. Plot 1903 illustrates a third region where energy is released during exothermic transitions, for example, effects of oxygen release to fuel thermal runaway where evolved O₂ is measured, e.g., using a mass spectrometer (not shown).

FIG. 19B is a graph 1950 illustrating additional NMC532 coin cell test results from the same experiment used to produce the graph results in FIG. 19A, namely, performed by a thermal analyzer and FTIR spectrometer. The waterplots at the front of the graph 1950, e.g., at 0-180° C. correlate to the plot 1901 shown in FIG. 19A. The increasing temperatures shown in waterplots in graph 1950 extend from front to back, and correspond to the temperature axis shown in FIG. 19A.

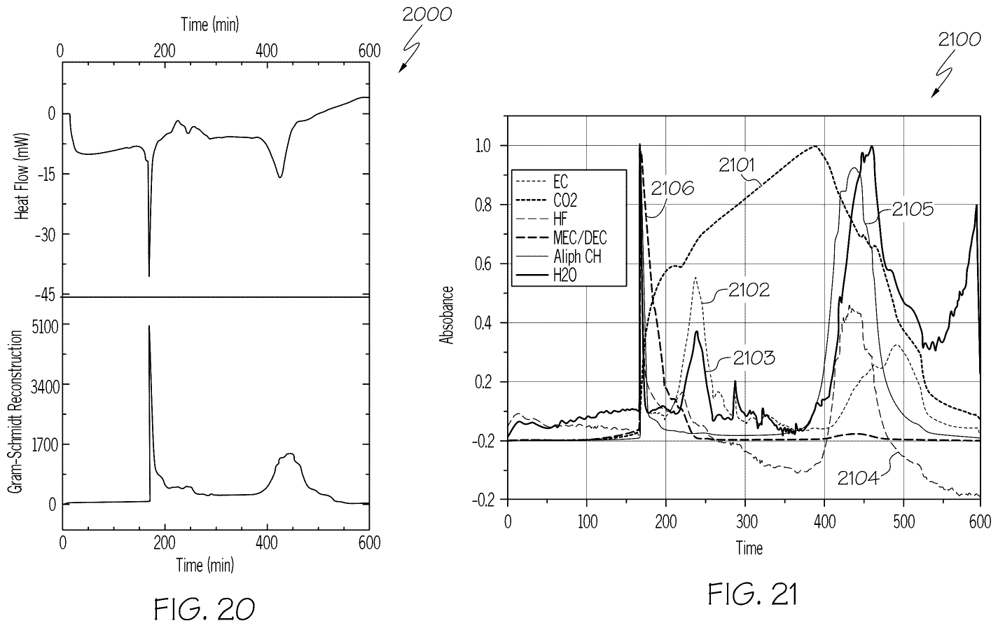

FIGS. 20 and 21 are graphs 2000, 2100 illustrating NMC532 coin cell DSC-FTIR results performed by a thermal analyzer, in accordance with some embodiments. FIGS. 20 and 21 may be similar to FIG. 19A except the comparable data is obtained from a different sample. A comparison of GSR curves include absorbance comparisons between electrolyte carbonate (EC) 2101, Co₂ 2102, H₂O 2103, HF 2104, Aliph C-H 2105, and methylene ethylene carbonate (MEC)/diethyl carbonate (DEC) 2106. FIGS. 20 and 21 show that reactions can be identified based on the products generated.

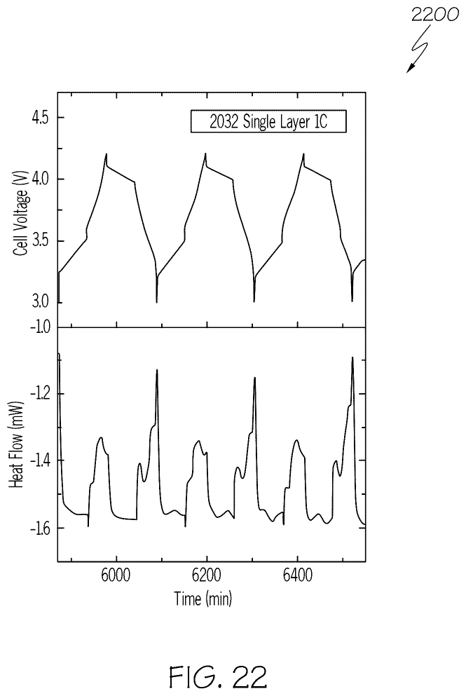

FIG. 22 illustrates a graph 2200 that shows heat flow generated by a 2032 single layer coin cell, for example, as it is charged and discharged in the calorimeter, using the cell capture container shown and described with reference to FIGS. 16A-16C, according to embodiments of the present inventive concept. With cycling, heat generation screening and overcharge limits can be provided.

A number of thermal test methods have been described, the experimental results of which are shown in the graphs described herein. A testing workflow using a thermal analyzer system described with reference to the various embodiments shown and described in FIGS. 1-16C will now be described. In a first step, a component material analysis is performed, which includes a building block of kinetic models and reaction enthalpies and degradation temperatures for cells components. In a second step, testing further is performed for an onset temperature and total energy release from full cells, which includes primary inputs for thermal modeling and which enables rapid comparison between different formulations. In a third step, further testing includes an identification of reactions through evolved gas analysis, resulting in an understanding of degradation mechanisms and an identification of hazardous gas species. Finally, in a fourth step, kinetic parameters are obtained to correlate with ARC testing or the like, which enables a prediction of full battery performance based on material testing and provides additional insight in cell degradation and pathways for improvements.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to explain the principles of the embodiments, the practical application or technical improvement over technologies found in the marketplace, or to enable others of ordinary skill in the art to understand the embodiments disclosed herein.

1. A battery material containment device, comprising:
a lid;
a housing case that is constructed and arranged to hold a battery material during a thermal analysis operation, the housing case including a threaded region for receiving and removably coupling with the lid to form a gas-tight seal and securing the battery material in the housing case;
at least one vent for providing a gas flow path for the release of gasses generated by the battery material in the housing case during the thermal analysis operation; and
a heat flow pathway between the battery material and a sensor that is maintained during the thermal analysis operation.

2. The battery material containment device of claim 1, further comprising an electrical insulator positioned on the battery material in the housing case for electrically insulating the battery material in the housing case.

3. The battery material containment device of claim 2, wherein the electrical insulator includes a mica insulator.

4. The battery material containment device of claim 1, wherein the housing holds a coin cell in which the battery material is positioned, and the heat flow pathway is maintained by preventing a coin cell deformation during an increase in pressure during the thermal analysis operation.

5. The battery material containment device of claim 4, wherein the coin cell includes a lid and a cup sealed together by a sealing gasket, and wherein the battery material is secured in the housing case past a sealing gasket melting point during the thermal analysis operation.

6. The battery material containment device of claim 1, wherein the battery material containment device is constructed and arranged in an instrument that provides for simultaneous ECA and EGA analyses of the thermal analysis operation.

7. The battery material containment device of claim 6, wherein the battery material containment device is constructed and arranged as an open container, wherein the housing case is constructed and arranged as a cup, wherein the battery material is positioned in a battery material housing that is secured in the cup, wherein the lid has a peripheral edge that clamps the battery material housing in the cup.

8. The battery material containment device of claim 7, further comprising a first pin extending from the lid and a second pin constructed and arranged as a low mass capsule chip pin on the battery material and extending from the at least one vent at a center of the lid, the second pin parallel to the first pin.

9. The battery material containment device of claim 1, wherein the at least one vent is a vent port in the lid.

10. The battery material containment device of claim 1, further comprising a controlled vent and sealing disk cap that is positioned over the battery material in the housing case and sealed in the housing case by the lid, wherein the at least one controlled vent is a vent port in the controlled vent and sealing disk cap.

11. The battery material containment device of claim 1, wherein the battery material containment device is constructed and arranged as an edge grip open container, wherein the lid is constructed and arranged as a low mass container lid, wherein the housing case is constructed and arranged as a low mass container cup, wherein the battery material is positioned in a coin cell that is secured in the pan cup and wherein the low mass container lid grips the coin cell at an edge of a bottom terminal.

12. The battery material containment device of claim 1, wherein the battery material containment device is constructed and arranged as a sealed container, wherein the lid is constructed and arranged as a sealed container lid, wherein the housing case is constructed and arranged as a sealed container cup, wherein the battery material is positioned in a coin cell that is secured in the sealed container cup, and wherein the sealed container lid applies a force against a top surface of the coin cell to secure the coin cell in the sealed container cup.

13. The battery material containment device of claim 1, further comprising:
An electrical contact spring in direct contact with an electrical terminal of the battery material;
an electrical feedthrough pin in electrical contact with a terminal of the battery material through the electrical contact spring.

14. The battery material containment device of claim 1, wherein the lid and the housing case are constructed and arranged as a coin cell housing, and the battery material secured in the coin cell housing comprises a cathode, an anode, and an electrically insulative separator between the cathode and the anode.

15. The battery material containment device of claim 14, further comprising a controlled vent and sealing disk cap having a burst disk and the at least one vent between the lip and the housing case for venting gas at a predetermined pressure.

16. The battery material containment device of claim 15, wherein the lid is constructed and arranged as a closure ring, wherein the panless coin cell housing includes a ledge below the threaded region, and where the battery material containment device further comprises:
an electrically insulative isolation and compression seal ring positioned on the ledge, a controlled vent and sealing disk cap having the burst disk positioned in the electrically insulative isolation and compression seal ring on the ledge; and
a spring between the electrically insulative isolation and compression seal and the battery material in the housing, the spring providing a pressure force to the battery material.

17. (canceled)

18. The battery material containment device of claim 1, further comprising an electrical post assembly connected to the battery material by positive and negative chip springs clamped by the lid, the electrical post assembly including an electrical insulation disk that separates the positive and negative chip springs from each other.

19. A system for analyzing thermal properties of a battery material, comprising:
a calorimetric sensor that measures a heat transfer with a battery material by detecting a temperature change when the battery material releases or absorbs heat during a thermal analysis operation;
a cell assembly that provides a housing for the battery material, is constructed and arranged to permit a reaction to occur by the battery material during a temperature increase, and to accommodate heat flow measurements for the battery material during the thermal analysis operation;
an insulated container over the cell assembly for insulating the cell assembly;
a lid assembly coupled to the insulated assembly,
the system further comprising either an electrical wire assembly housed in the lid assembly that make electrical contact to a positive and negative terminal of the battery material or a hair wire ECA assembly for coupling between the battery material in the cell assembly and a differential scanning calorimetry (DSC) device through a bottom of the cell assembly; and
a gas flow path from the lid assembly.

20-33. (canceled)

34. A calorimetry system for analyzing thermal properties of a battery material, comprising:
a battery material containment device constructed and arranged to secure a coin cell or a source of battery material; and
a cell assembly that provides a housing that accommodates and secures the battery material containment device during a combination of thermal analysis and electrical measurement operations, wherein the thermal analysis operations include a coin cell or source of battery material exposed to a temperature of up to 600° C.;.

35-36. (canceled)
